(19) United States
(12) Patent Application Publication
(10) Pub. No.: US 2024/0207653 A1
(43) Pub. Date: Jun. 27, 2024

(54) IMAGING-BASED TARGETING FOR TRANS-CRANIAL FOCUSED ULTRASOUND SYSTEMS

(71) Applicant: Sanmai Technologies, PBC, Sunnyvale, CA (US)

(72) Inventors: Christopher DAFT, Tucson, AZ (US); Ashish PARIKH, Los Altos, CA (US); Tomokazu SATO, Minneapolis, MN (US); Bicheng WU, Palo Alto, CA (US); Timothy MULLEN, Hidden Meadows, CA (US)

(21) Appl. No.: 18/478,906

(22) Filed: Sep. 29, 2023

Related U.S. Application Data
(60) Provisional application No. 63/435,158, filed on Dec. 23, 2022.

(51) Int. Cl. A61N 7/00

(52) U.S. Cl. CPC A61N 7/00; A61N 2007/0026; A61N 2007/0078; A61N 2007/0091

(57) ABSTRACT

A trans-cranial Focused Ultrasound System (tFUS) apparatus employs an ultrasonic stimulation beam that is guided by ultrasonic imaging or optical imaging to optimize a response of a subject during a procedure. The disclosed tFUS apparatus is operable without requiring either an MRI or a CT scan. The use of multiple transducers or transducer elements, including contralateral transducers, the use of ultrasonic and/or optical harmonics, the use of temporal windows, the use of a head atlas, motion tracking, LiDAR, and the use of aberration correction parameters may all be employed individually or in combination.

### CROSS-REFERENCE TO RELATED APPLICATIONS

This application claims the priority benefit of U.S. Provisional Patent Application titled, “APPARATUS FOR tFUS STIMULATION GUIDED BY IMAGING,” filed on December 23, 2022, having application Ser. No. 63/435,158. The subject matter of this related application is hereby incorporated herein by reference in its entirety.

### BACKGROUND

### Field of the Various Embodiments

Embodiments disclosed herein generally relate to trans-cranial focused ultrasound systems and, more specifically, to imaging-based targeting for trans-cranial focused ultrasound systems.

### Description of the Related Art

Medical professionals employ various brain stimulation techniques to treat a great number of neurological and psychiatric disorders. These include non-invasive techniques that stimulate targeted portions of the brain; operators employ such techniques in lieu of techniques that require surgical placement of equipment on the brain of a subject. A trans-cranial focused ultrasound system (tFUS) enables operators to perform non-invasive techniques by transmitting low-intensity ultrasound waves into the brain of a subject, thereby delivering ultrasonic energy upon deep brain regions of the subject. A given tFUS guides a stimulation beam of ultrasonic energy to a target location within the brain region of the subject based on imaging information of the brain. Various imaging methods acquire imaging of the brain of a subject to enhance the efficacy of the tFUS delivering the ultrasonic stimulation to the target location. Such methods maximize the sound pressure, delivered by the tFUS via the ultrasonic stimulation, that reaches the target structure while minimizing any associated leakage sound pressure from the ultrasonic stimulation that transmits to non-target regions of the brain.

Two categories of conventional tFUS include systems that acquire and use real-time imaging during the procedure, as well as systems that employ neuro-navigation on pre-procedure imaging. For example, systems that employ real-time imaging include a tFUS that acquires near real-time magnetic resonance imaging (MRI) during a procedure on the brain of a subject. In this approach, the tFUS includes an MRI-compatible ultrasound transducer that stimulates the brain while the subject is inside an MRI scanner. Such systems enable medical experts to observe the effect of the stimulation a short time after the tFUS provides the stimulation, using functional MRI techniques for the imaging, such as blood-oxygen-level dependent (BOLD) contrast or arterial spin labeling (ASL).

In another example, a tFUS that employs neuro-navigation acquires a set of pre-procedure images of the brain anatomy of the subject via an MRI scanner. A volume representing the brain of the subject is fed into a neuro-navigation system that computes a relationship between a coordinate system of the scanned volume, a coordinate system of the patient, and a coordinate of the tFUS. The neuro-navigation system identifies a location and orientation for placement of the transducer of the tFUS onto the subject such that the focus of the stimulation beam intersects with the target location. An example of a neuro-navigation system includes a computing device and two infrared cameras for stereoscopic evaluation of the coordinate system of the patient.

At least one drawback of systems that include a conventional tFUS is that such systems require the use of expensive imaging techniques to effectively deliver ultrasonic energy. For example, an MRI-compatible tFUS is configured to function with MRI scanners; however, such MRI scanners are expensive, complicated to operate, and require separate expertise to control. The cost and skill requirements associated with the MRI scanner thereby limit the availability the of an MRI-compatible tFUS in various brain procedures. The alternative usage of neuro-navigation systems with a conventional tFUS similarly adds additional expensive and complex equipment to a given procedure, as the neuro-navigation system requires additional medical experts to operate before an operator can properly set up the components of the conventional tFUS.

In view of the foregoing, there is a need in the art for more efficient and cost-effective targeting structures and methods for ultrasonic stimulation.

### SUMMARY

Various embodiments disclose a trans-cranial focused ultrasound system (tFUS) apparatus comprising a set of at least two transducers that produce ultrasonic stimulation and ultrasonic guidance means or optical guidance means that guide the ultrasonic stimulation.

Other embodiments of the present disclosure set forth a method comprising determining positioning information associated with a set of at least two transducers that produce ultrasonic guidance information, acquiring information for a subject proximate to the set of at least two transducers, identifying a target position based on the positioning information and the imaging information, and providing, by at least one of the set of at least two transducers, an ultrasonic beam to the target position.

At least one technological advantage of the disclosed imaging-based targeting for trans-cranial focused ultrasound systems relative to the prior art is that, with the disclosed apparatus and techniques, a trans-cranial focused ultrasound system is provided accurate imaging and delivers focused ultrasonic stimulation to a target area based on the improved imaging. In particular, the combination of transducers and additional means to guide the ultrasonic stimulation to provide ultrasonic stimulation to the target area. Further, as the disclosed system incorporates imaging data from sources alternative to MRI and CT (computerized tomography) scans, a tFUS adapted to deliver ultrasonic energy based on the imaging data provides cost-effective alternatives to systems that require expensive MRI scanners or neuro-navigation systems to accurately guide a tFUS. These technical advantages provide one or more technological advancements over prior art approaches.

### BRIEF DESCRIPTION OF THE DRAWINGS

So that the manner in which the above recited features of the various embodiments can be understood in detail, a more particular description of the inventive concepts, briefly summarized above, may be had by reference to various embodiments, some of which are illustrated in the appended drawings. It is to be noted, however, that the appended drawings illustrate only typical embodiments of the inventive concepts and are therefore not to be considered limiting of scope in any way, and that there are other equally effective embodiments.

FIG. 1A is a schematic diagram illustrating a prior art trans-cranial focused ultrasound system (tFUS).

FIG. 1B illustrates an acoustic energy profile and acoustic parameters associated with the prior art tFUS.

FIG. 2 is a graph illustrating a prior art group average fast Fourier transform electroencephalographic (EEG) frequency spectra.

FIG. 3 illustrates a prior art low-intensity tFUS system using neuro-navigation.

FIG. 4 illustrates an ultrasound transducer of a tFUS system coupled to a head of a subject, according to various embodiments.

FIG. 5 illustrates a pair of ultrasound transducers of a tFUS of FIG. 4 coupled to a head of a subject, according to various embodiments.

FIG. 6 illustrates an exemplary operation of the tFUS of FIG. 4 scanning a head volume, according to various embodiments.

FIG. 7 illustrates another exemplary operation of the tFUS of FIG. 4 scanning a head volume in conjunction with a separate device, according to various embodiments.

FIG. 8 illustrates another exemplary operation of the tFUS of FIG. 4 including a transducer having an optical registration feature, according to various embodiments.

FIG. 9 sets forth a flow chart of method steps for providing ultrasonic stimulation to a target location of a brain of a subject using the tFUS of FIG. 4, according to various embodiments.

### DETAILED DESCRIPTION

In the following description, numerous specific details are set forth to provide a more thorough understanding of the various embodiments. However, it will be apparent to one skilled in the art that the inventive concepts may be practiced without one or more of these specific details.

### Trans-Cranial Focused Ultrasound System Overview

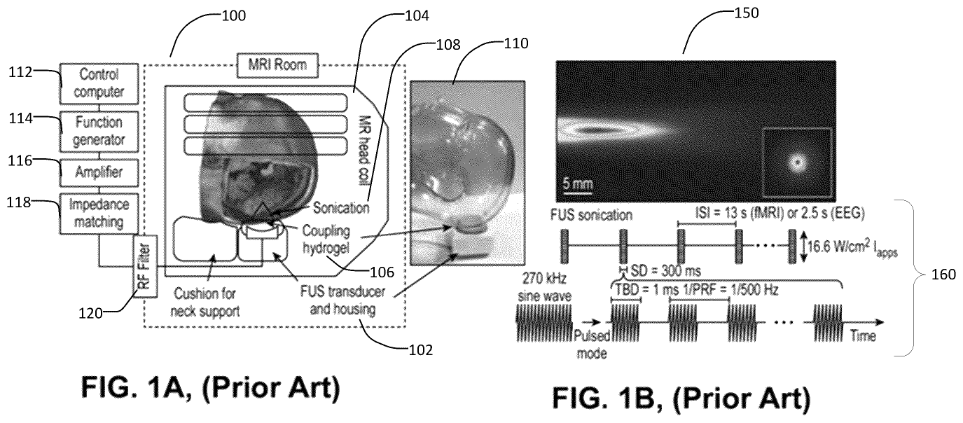

FIG. 1A is a schematic diagram illustrating a prior art trans-cranial focused ultrasound system (tFUS) 100. FIG. 1B illustrates an acoustic energy profile 150 and acoustic parameters 160 associated with the prior art tFUS 100. FIGS. 1A and 1B are presented in “Transcranial focused ultrasound stimulation of human primary visual cortex,” by Wonhye Lee et al., which is hereby incorporated by reference. FIG. 1A illustrates a prior art tFUS application including an tFUS transducer and housing 102 positioned inside of a magnetic resonance (MR) head coil 104, together with a corresponding mannequin image 110. The tFUS system also includes a coupling hydrogel 106 and a sonication region 108. A control computer 112 controls a function generator 114, an amplifier 116 and an impedance matching network 118 that connects through an RF filter 120 to the FUS transducer. FIG. 1B illustrates a prior art acoustic energy profile 150 (lateral and axial) with associated acoustic parameters 160, including SD (sonication duration), ISI (inter-stimulation interval), together with TBD (tone burst duration) and PRF (pulse-repetition frequency).

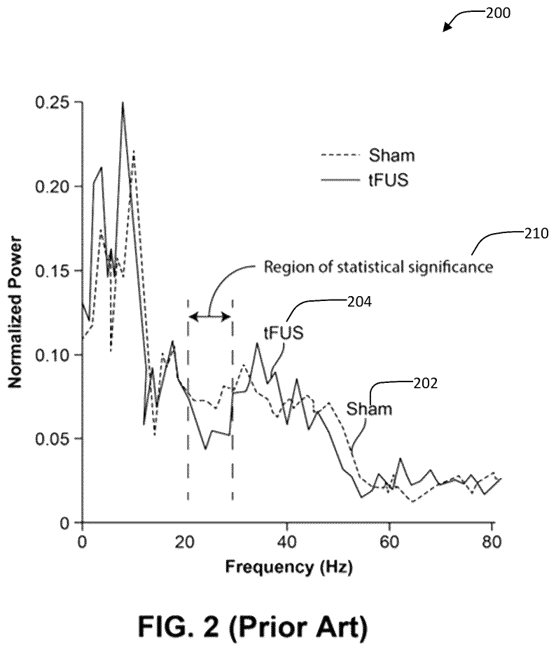

FIG. 2 is a graph 200 illustrating a prior art group average fast Fourier transform electroencephalographic (EEG) frequency spectra. FIG. 2 is a graph presented in a paper by Wynn Legon et al, “Neuromodulation with single-element transcranial focused ultrasound in human thalamus,” Human Brian Mapping, 39(5), 1995-2006. This paper includes group average FFT (fast Fourier transform) frequency spectra, shown as normalized power vs frequency with a Sham 202 and a tFUS response 204. A region of statistical significance 210 is shown. The Sham response 202 is the response achieved when the stimulation beam is turned off, but all other measurement parameters remain unchanged.

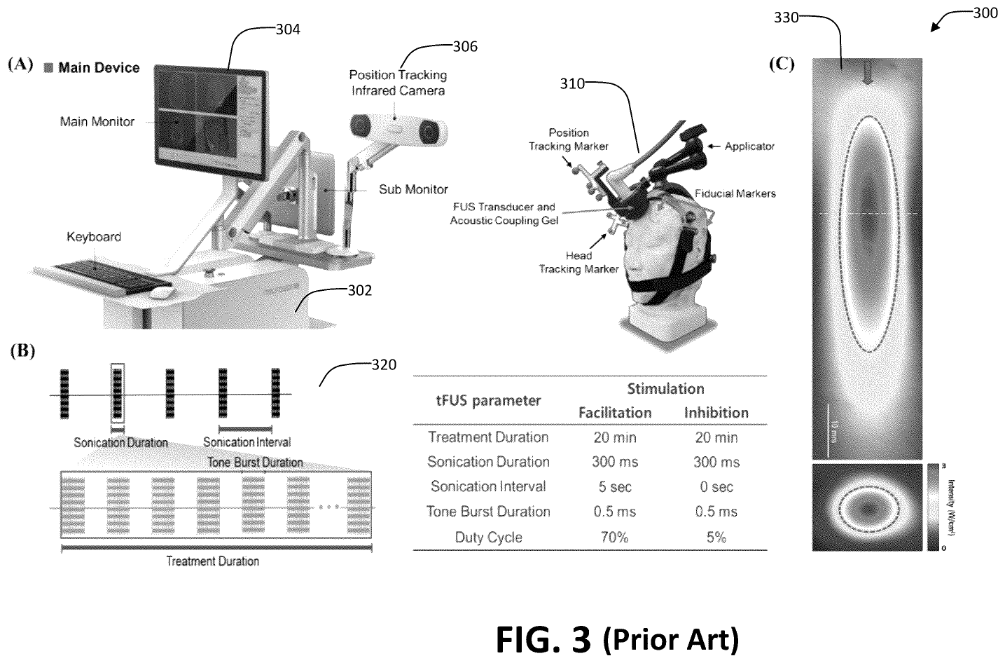

FIG. 3 illustrates a prior art low-intensity tFUS system 300 using neuro-navigation. FIG. 3 illustrates a low-intensity tFUS system 300 presented in “Neuromodulation Using Transcranial Focused Ultrasound on the Bilateral Medial Prefrontal Cortex,” by Young Goo Kim et al., which is hereby incorporated by reference. The low-intensity tFUS system 300 includes a PC 302, a monitor 304, infra-red cameras 306, and an FUS transducer 310. FIG. 300 also includes a schematic diagram 320 of sonication parameters (e.g., sonication duration, sonication interval, treatment duration, tone burst duration, duty cycle, etc.) in group (B). The PC 302 controls the operation of the FUS transducer 310 to provide the stimulation in accordance with the sonication parameters, creating the acoustic intensity profile 330 in lateral and axial planes in group (C).

### Imaging-Based Targeting for Trans-Cranial Focused Ultrasound Systems

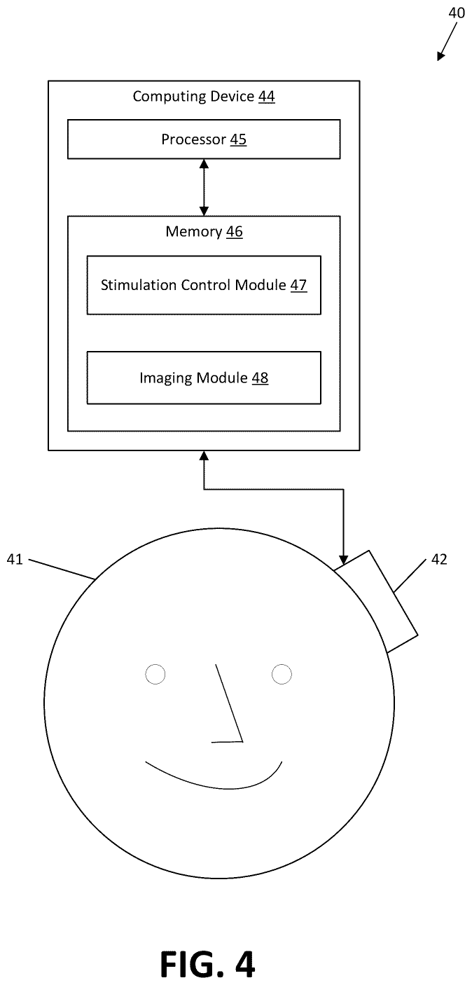

FIG. 4 illustrates an ultrasound transducer of a tFUS system 40 coupled to a head 41 of a subject, according to various embodiments. The tFUS system 40 in accordance with one or more embodiments includes a single ultrasound transducer 42 that is coupled to the head 41. The ultrasound transducer 42 communicates with a computing device 44. In various embodiments, the ultrasonic transducer 42 may include many elements. The computing device 44 includes, without limitation, a processor 45 and a memory 46. The memory 46 includes, without limitation, a stimulation control module 47 and an imaging module 48.

In operation, the stimulation control module 47 causes the transducer 42 to provide an ultrasonic stimulation beam to a target location within the head 41 of the subject. The imaging module 48 provides a form of imaging other than MRI or CT (e.g., acquiring images from one or more cameras, or acquiring images from the ultrasonic transducer 42) that the stimulation control module 47 uses to identify locations within the head 41 to guide the ultrasound transducer 42 in directing the ultrasonic stimulation beam.

The computing device 44 communicates with the ultrasound transducer 42 to control the ultrasonic stimulation beam generated by the ultrasonic transducer 42. In various embodiments, the computing device 44 is a desktop, laptop, mobile device (e.g., mobile phone, tablet, etc.), wearable device, or a component of various augmented reality (AR) devices (e.g., a console), communications systems, and so forth.

The processor 45 can be any suitable processor, such as a central processing unit (CPU), a graphics processing unit (GPU), an application-specific integrated circuit (ASIC), a field programmable gate array (FPGA), a digital signal processor (DSP), and/or any other type of processing unit, or a combination of different processing units, such as a CPU configured to operate in conjunction with a GPU. In general, the processor 45 can be any technically feasible hardware unit capable of processing data and/or executing software applications.

The memory 46 can include a random-access memory (RAM) module, a flash memory unit, or any other type of memory unit or combination thereof. The processor 45 is configured to read data from and write data to memory 46. In various embodiments, the memory 46 includes non-volatile memory, such as optical drives, magnetic drives, flash drives, or other storage. In some embodiments, separate data stores, such as an external included in a separate network (“cloud storage”) can supplement the memory 46. The stimulation control module 47 and/or the imaging module 48 within the memory 46 can be executed by the processor 45 to implement the overall functionality of the computing device 44 and, thus, to coordinate the operation of the tFUS system 40 as a whole. In various embodiments, an interconnect bus (not shown) connects the processor 45, the memory 46, and an input/output (I/O) device interface (not shown) that connects the computing device 44 to ultrasonic transducer 42.

In various embodiments, the stimulation control module 47 identifies a target area for an ultrasonic stimulation beam and controls the ultrasonic transducer 42 to generate the ultrasonic stimulation beam. In various embodiments, the stimulation control module 47 acquires positioning information and/or imaging information provided by the imaging module 48 to accurately map a portion of the head 41 of the subject and identify specific locations, including the location of the ultrasonic transducer 42, as well as one or more target locations within a three-dimensional space bounded by the head 41. The stimulation control module 47 uses the positioning information and the imaging information to direct ultrasonic stimulation beams generated by the ultrasonic transducer 42.

Additionally or alternatively, in some embodiments, the stimulation control module 47 provides various ultrasonic guidance means by detecting and/or verifying a skull aberration correction, where the stimulation control module 47 performs such corrections during a given stimulation procedure to ameliorate the negative effects of skull aberrations of the head 41. In some embodiments, the stimulation control module 47 determines the local thickness of skull layers of the head 41. For example, the stimulation control module 47 can control the ultrasonic transducer 42 to generate ultrasonic stimulation beams and measure ultrasonic echoes within the head 41. The stimulation control module 47 can then determine differences in echo harmonics to identify any skull aberrations. In some embodiments, the stimulation control module 47 also uses ultrasonic data to identify ventricles and blood vessels within the head 41.

In various embodiments, the imaging module 48 generates the imaging information using one or more guidance means. In some embodiments, the imaging module 48 acquires ultrasonic data via one or more ultrasonic means, including one or more ultrasonic transducers 42. Additionally or alternatively, the imaging module 48 acquires optical data via optical guidance means, including one or more cameras, such as infrared cameras, LiDAR scanners, and/or other cameras included in one or more devices (e.g., a LIDAR scanner in a mobile phone). In some embodiments, the imaging module 48 suppresses clutter at the fundamental frequency by implementing a non-linear imaging mode. In such instances, the computing device 44 receives harmonic energy; for example, the computing device 44 via the ultrasonic transducer 42 or a separate sensor (not shown) can selectively receive a second harmonic associated with the ultrasonic stimulation beam.

Additionally or alternatively, the imaging module 48 can employ various tracking mechanisms to estimate the relative locations of the transducer 42 and the head 41 of the subject. For example, the imaging module 48 can employ a speckle tracking algorithm, or another motion estimation algorithm, to generate positioning information related to the change in the relative position of the transducer 42 to the head 41. In different embodiments, the imaging module 48 provides guidance for the stimulation control module 47 using strain-sensitive contrast, such as acoustic radiation force imaging (ARFI).

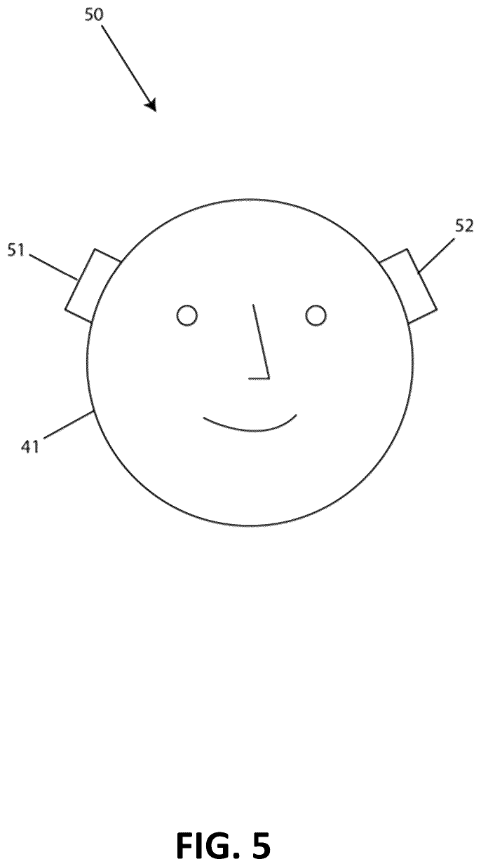

FIG. 5 illustrates a pair of ultrasound transducers 51, 52 of a tFUS system 50 coupled to a head 41 of a subject, according to various embodiments. In operation, the pair of transducers 51, 52 each produce insonifying volumes when producing ultrasonic stimulation beams. The respective insonifying volumes intersect within the volume formed by the head 41, providing advantageous imaging performance.

A set of transducers, including at least the transducers 51, 52, are attached to the head 41 of the subject. In some embodiments, the tFUS system 50 includes three or more transducers (not shown). Each of the three or more transducers generates an insonifying volume within a portion of the head 41 when activated by the stimulation control module 47. In various embodiments, each of the three or more insonifying volumes intersect at specific locations, such as at a target location within the head 41.

In some embodiments, at least one of the transducers 51, 52 enables transmission imaging (e.g., generating maps of ultrasonic waves within the volume of the head 41). In such instances, the imaging module 48 generates the imaging from the data generated by the ultrasonic waves and provides ultrasonic guidance to the stimulation control module 47. In various embodiments, the tFUS system 50 employs different frequencies for stimulation and guidance. For example, the stimulation control module 47 controls one of the transducers (e.g., the transducer 51 acting as a “guidance transducer”) to generate ultrasonic data for imaging by generating an ultrasonic frequency that is approximately twice a center frequency of the ultrasonic stimulation beam that a separate transducer (e.g., the transducer 52 acting as a “stimulation transducer”) creates. In some embodiments, the stimulation transducer 52 and the guidance transducer 51 are positioned on the temporal windows of the head 41.

In various embodiments, one or more transducers are positioned at contra-lateral positions on the head 41. In such instances, the imaging module 48 receives echo signals from the respective contra-lateral transducers and generates ultrasonic data based on the echo signals. In some embodiments, the imaging module 48 generates ultrasonic data that includes an observation of differences between signal strengths of the fundamental frequency and the harmonic frequencies. Such ultrasonic data is used by the stimulation control module 47 to optimize guidance of the ultrasonic stimulation beam. Additionally or alternatively, in some embodiments, the stimulation control module 47 uses the contra-lateral transducers to minimize deleterious effects of refraction, or other aberration effects in the head 41.

In various embodiments, at least one of the transducers is a transmitting transducer positioned at the back of the head 41. In such instances, the stimulation control module 47 uses the transducer positioned at the back of the head 41 to facilitate stimulation of structures within an approximately conical volume radiating from the transducer. For example, the transducer can generate a conical volume with a small angular range; a separate transducer is positioned at the temporal window to receive a signal and is centered to receive signals at twice the frequency produced by the transducer positioned at the back of the head. In this manner, the separate transducer receives various signals associated with angular scattering created by the transmission from the transducer positioned at the back of the head.

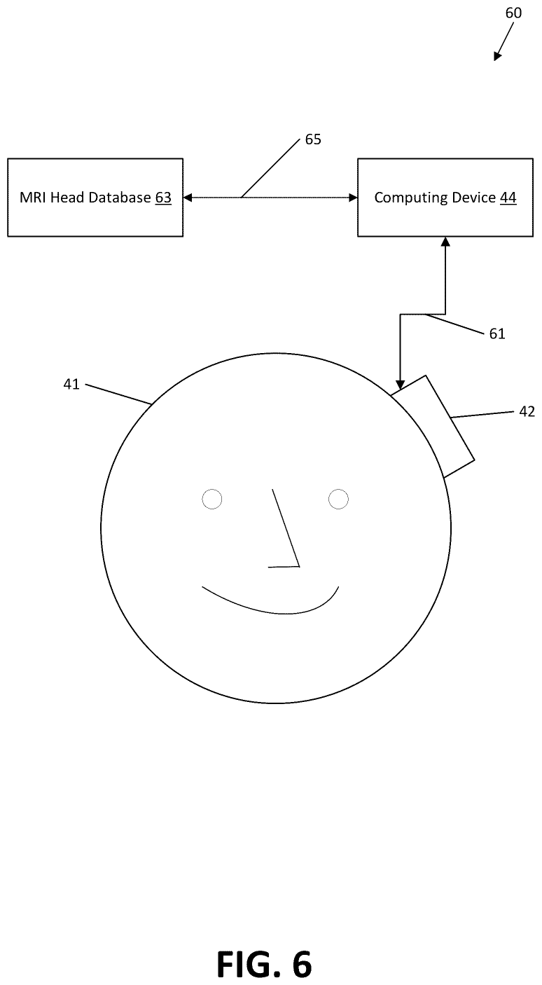

FIG. 6 illustrates an exemplary operation of the tFUS system of FIG. 5 scanning a head volume, according to various embodiments. As shown, and without limitation, the tFUS setup 60 includes the ultrasound transducer 42 contacting the head 41 of the subject, the computing device 44, and an MRI head database 63.

In operation, the computing device 44 establishes a connection 61 to the ultrasound transducer 42. The imaging module 48 of the computing device 44 acquires imaging information from the transducer 42. The computing device 44 also establishes a separate connection 65 to an MRI head database 63 that includes a database of MRI scans. The imaging module 48 or the stimulation control module 47 compares a head ultrasound volume generated by the imaging module 48 with the MRI cands in the MRI head database 63.

In various embodiments, the transducer 42 is in communication via ultrasound sending and receiving circuits (e.g., the connection 61) to a processor 62 that compares results with an MRI head database 63. The imaging module 48 receives ultrasonic data and compares the ultrasonic data or corresponding imaging information with an atlas of MRI or CT scans of heads from the MRI head database 63. Identification of similar imaging enables the computing device 44 to make identification of the target structure more reliable. In some embodiments, the imaging module 48 employs an associative look-up, incorporating a-priori information. In some embodiments, the look-up is informed by additional information, such as the position of vessels detected by pulsed Doppler imaging, and/or the position of ventricles detected by harmonic grayscale imaging. Other cataloged data may be used as a-priori information.

In some embodiments, the computing device 44 blocks associated procedures from being performed on the subject until the imaging module 48 determines an adequate correlation between the imaging information acquired during the procedure and a head atlas generated from imaging information is achieved. For example, if the transducer 42 is incorrectly positioned on the head 41, the imaging module 48 determines that the set of landmarks cannot be found at the expected locations. In such instances, look-ups to historical data, such as maps or atlases stored in the MRI head database 63 will fail. The stimulation control module 47 responds to the failed lookup by stopping (or refusing to start) the stimulation procedure. The stimulation control module 47 can also signal that repositioning of the transducer 42 and a successful correlation is necessary.

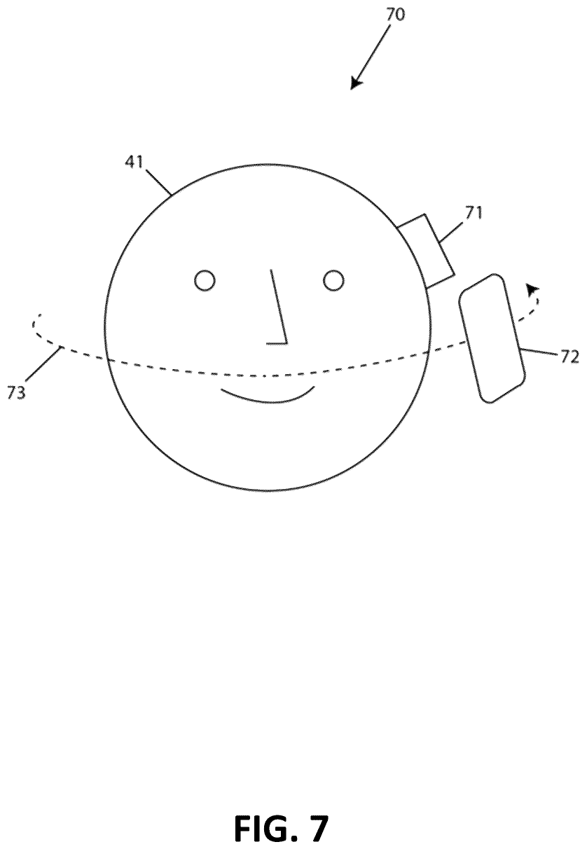

FIG. 7 illustrates another exemplary operation of the tFUS system of FIG. 5 scanning a head volume in conjunction with a separate device, according to various embodiments. As shown, and without limitation, the tFUS setup 70 includes a transducer 71 contacting the head 41 of a subject and a mobile imaging device 72.

In operation, the mobile imaging device 72 traverses along a path 73 around the head 41. The mobile imaging device 72 employs LIDAR (Light Detection and Ranging) or other types of photographic imaging to measure the geometry of the head 41. In such instances, the imaging module 48 of the computing device 44 receives optical data from the mobile imaging device 72 and employs optical guidance for the stimulation control module 47.

In various embodiments, the mobile imaging device 72 is a mobile phone equipped with LIDAR and/or photographic capability. In some embodiments, the mobile imaging device 72 includes one or more MEMs (Micro-Electromechanical Systems) sensors that track the position and orientation of the mobile imaging device 72. Additionally or alternatively, the mobile imaging device 72 includes an imaging application (not shown) that generates a mesh representation of the head 41 based on imaging data acquired by the mobile imaging device 72. In some embodiments, the mobile imaging device 72 provides the mesh representation and/or a corresponding head scan to the imaging module 48 of the computing device 44. The imaging module 48 uses the mesh representation and/or head scan to adapt a default representation generated from an average brain (e.g., averaging data acquired from the Montreal Neurological Institute) to represent the brain of the subject.

In some embodiments, the computing device 44 communicates with the mobile imaging device 72 to identify and optically verify that the transducer 42 is contacting the head 41 at a target position.

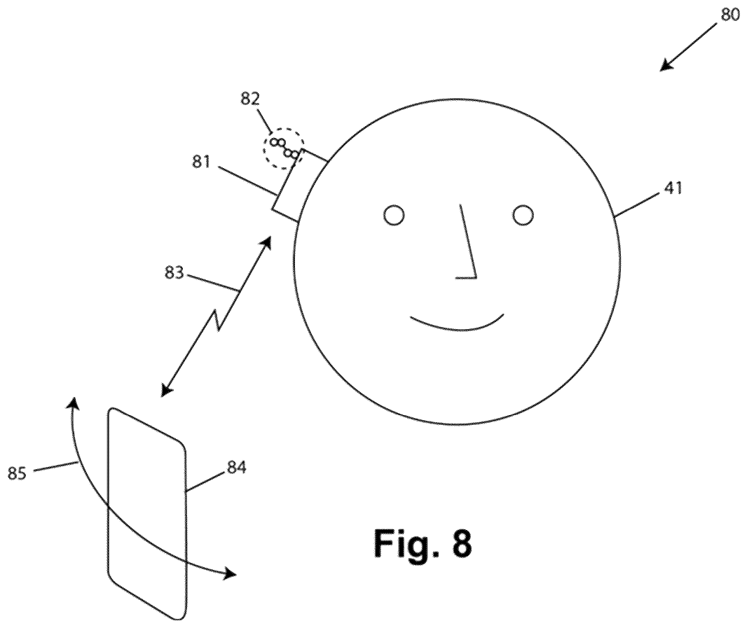

FIG. 8 illustrates another exemplary operation of the tFUS system of FIG. 5 including a transducer having an optical registration feature, according to various embodiments. As shown, and without limitation, the tFUS setup 80 includes a transducer 81 and optical feature 82 contacting the head 41 of a subject and a mobile imaging device 84.

In operation, the transducer 81 is in communication 83 with the mobile imaging device 84. As the mobile imaging device 84 traverses along path 85, the mobile imaging device 84 detects the position and orientation of the optical feature 82. Based on the determination of the position and orientation of the optical feature 82, the mobile imaging device 84 and/or the imaging module 48 determines the position and orientation of the transducer 81 and controls beam steering and focusing using an adapted brain model. In some embodiments, the mobile imaging device 84 and/or the imaging module 48 are adaptable to determine the position and orientation of the transducer 81 based on relative motion between the transducer 81 the head 41.

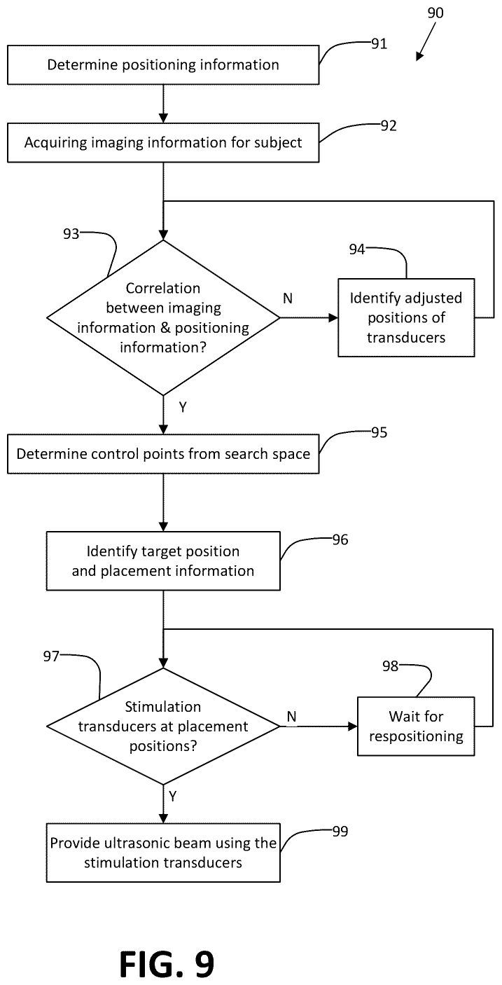

FIG. 9 sets forth a flow chart of method steps for providing ultrasonic stimulation to a target location of a brain of a subject using the tFUS of FIG. 4, according to various embodiments. Although the method steps are described with reference to the embodiments of FIGS. 4-8, persons skilled in the art will understand that any system configured to implement the method steps, in any order, falls within the scope of the present disclosure.

As shown in FIG. 9, the method 900 begins at step 91, where the computing device 44 acquires positioning information. In various embodiments, the imaging module 48 operating on the computing device 44 acquires positioning information related to the positions of one or more ultrasonic transducers 42 contacting a head 41 of a subject. In some embodiments, the computing device 44 communicates with the mobile imaging device 72 to identify and optically verify that the transducer 42 is contacting the head 41 at a target position.

In some embodiments, the imaging module 48 derives positioning information from ultrasound data collected by one or more transducers 42. For example, a guidance transducer 51 can initially generate ultrasonic frequencies that are captured by one or more transducers 51, 52. The stimulation control module 47 and/or the imaging module 48 can then determine the positions of the one or more transducers 51, 52 relative to the head 41. Alternatively, in some embodiments, the imaging module 48 acquires optical data from an optical sensor, such as images acquired by the mobile imaging device 72. In such instances, the imaging module 48 can determine the shape of the head 41 and the positions of the one or more transducers 51, 52 relative to the head 41.

At step 92, the computing device 44 acquires imaging information for the subject. In various embodiments, the imaging module 48 acquires and/or generates imaging information using one or more guidance means. In some embodiments, the imaging module 48 acquires ultrasonic data via one or more ultrasonic means, including one or more ultrasonic transducers. In some embodiments, at least one of transducers 51, 52 enables transmission imaging (e.g., generating maps of ultrasonic waves within the volume of the head 41). In such instances, the imaging module 48 generates the imaging from the data generated by the ultrasonic waves and provides ultrasonic guidance to the stimulation control module 47.

Additionally or alternatively, the imaging module 48 acquires optical data via optical guidance means, including one or more cameras, such as infrared cameras, LIDAR scanners, and/or other cameras included in one or more devices (e.g., a LiDAR scanner in a mobile phone). In some embodiments, the mobile imaging device 72 includes one or more MEMs (Micro-Electromechanical Systems) sensors that track the position and orientation of the mobile imaging device 72. In some embodiments, the mobile imaging device 72 includes an imaging application that generates a mesh representation of the head 41 based on imaging data acquired by the mobile imaging device 72. In some embodiments, the mobile imaging device 72 provides the mesh representation and/or a corresponding head scan to the imaging module 48. The imaging module 48 uses the mesh representation and/or head scan to adapt a default representation generated from an average brain to represent the brain of the subject.

At step 93, the computing device 44 determines whether there is a sufficient correlation between the imaging information and the positioning information. In various embodiments, the stimulation control module 47 determines whether the imaging module determined an adequate correlation between the acquired imaging information and a head atlas generated from the imaging information. When the stimulation control module 47 determines that the correlation is below a pre-defined threshold, the stimulation control module 47 blocks procedures from being performed on the subject and proceeds to step 94. Otherwise, the stimulation control module 47 determines that the correlation at or above the pre-defined threshold and proceeds to step 95. For example, if the transducer 42 is incorrectly positioned on the head 41, the imaging module 48 determines that a set of landmarks cannot be found at the expected locations. In such instances, look-ups to historical data, such as maps or atlases stored in the MRI head database 63 will fail and result in a low correlation value. The stimulation control module 47 responds to the failed lookup by stopping (or refusing to start) the stimulation procedure. The stimulation control module 47 can also signal that repositioning of the transducer 42 and a successful correlation is necessary.

At step 94, the computing device 44 identifies the adjusted positions of the one or more transducers 42 contacting the head 41 of the subject. In various embodiments, the stimulation control module 47 signals that repositioning of the transducer 42 and a successful correlation is necessary before proceeding. An operator can then respond to the signal by adjusting the positions of the one or more transducers 42. In various embodiments, the imaging module 48 employ various tracking mechanisms to estimate the adjusted positions of the one or more transducers 42. For example, the imaging module 48 can employ a speckle tracking algorithm, or another motion estimation algorithm, to generate positioning information related to the change in the relative position of the transducer 42 to the head 41. In different embodiments, the imaging module 48 provides guidance for the stimulation control module 47 using strain- sensitive contrast, such as acoustic radiation force imaging (ARFI). Upon identifying the adjusted positions of the one or more transducers 42, the stimulation control module 47 returns to step 93 to determine whether there is adequate correlation between the positioning information corresponding to the adjusted positions of the transducers 42 and the imaging information that includes the head atlas for the head 41.

At step 95, the computing device 44 determines control points for a search space. In various embodiments, the stimulation control module 47 uses various targeting algorithms to identify target locations within the head 41 of the user. In some embodiments, the stimulation control module 47 obtains starting points and/or ranges of a search space associated with known head atlases.

At step 96, the computing device 44 identifies target position and placement information. In various embodiments, the stimulation control module 47 acquires imaging information provided by the imaging module 48 to accurately map a portion of the head 41 of the subject and identify specific locations, including one or more target locations within a three-dimensional space bounded by the head 41, to direct ultrasonic stimulation beams generated by the ultrasonic transducer 42. In various embodiments, the stimulation control module 47 executes a targeting algorithm using the initial starting points or ranges of the search space to determine one or more target locations within the head 41 of the subject that are to receive ultrasonic stimulation from one or more ultrasonic stimulation beams generated by the one or more transducers 42.

At step 97, the computing device 44 determines whether the stimulation transducers are at the applicable placement positions. In some embodiments, the stimulation control module 47 executes the targeting algorithm, where the stimulation control module 47 uses results of the targeting algorithm (e.g., zero target locations) to determine whether the positions of the transducers require repositioning. When the stimulation control module 47 determines that the one or more transducers 42 are at the proper placement positions, the stimulation control module 47 proceeds to step 99. Otherwise, the stimulation control module 47 determines that the one or more transducers 42 are not at the proper placement positions and proceeds to step 68.

At step 98, the computing device 44 waits for the transducers to be repositioned. In various embodiments, the stimulation control module 47 signals that repositioning of the transducer 42 and is necessary before proceeding to provide the ultrasonic beam. An operator can then respond to the signal by adjusting the positions of the one or more transducers 42. In various embodiments, the imaging module 48 employ various tracking mechanisms to estimate the adjusted positions of the one or more transducers 42. Upon identifying the new positions of the one or more transducers 42, the stimulation control module 47 returns to step 97 to determine whether the stimulation transducers are at the proper placement positions.

At step 99, the computing device 44 provides an ultrasonic beam using the stimulation transducers. In various embodiments, the stimulation control module 47 directs ultrasonic stimulation beams generated by the ultrasonic transducer 42 such that the focus of the stimulation beam intersects with the identified target locations within the head 41 of the subject. In various embodiments, the stimulation control module 47 can modify one or more parameters of the ultrasonic stimulation beams produced by the stimulation transducers. For example, the stimulation control module 47 can modify multiple parameters, including the amplitude, frequency, duty cycle, and so forth.

In sum, a computing device includes a stimulation control module that causes one or more transducers to provide ultrasonic stimulation beams to a target location within a subject. The computing device also includes an imaging module that provides imaging information (e.g., images in forms other than other than MRI or CT, such as by acquiring images from one or more cameras, etc.) that the stimulation control module uses to identify locations within the head to guide the ultrasound transducers in directing ultrasonic stimulation beams.

The apparatus and methods previously described are used in various embodiments for (i) the method of placement and adjustments for placement of transducers; (ii) the initial placement of transducers; (iii) obtaining starting points or ranges of a search space in support of a targeting algorithm. Accordingly, the results of ultrasonic imaging or optical imaging are used to obtain control points in a search space, providing means for the targeting algorithm to operate on the control points. In some embodiments, external tasks or stimuli are used to adjust phasing, corrections, and/or targeting. Examples include auditory or visual activations, the use of language, or the use of one or more motors.

At least one technological advantage of the disclosed imaging-based targeting for trans-cranial focused ultrasound systems relative to the prior art is that, with the disclosed apparatus and techniques, a trans-cranial focused ultrasound system is provided accurate imaging and delivers focused ultrasonic stimulation to a target area based on the improved imaging. In particular, the combination of transducers and additional means to guide the ultrasonic stimulation to provide ultrasonic stimulation to the target area. Further, as the disclosed system incorporates imaging data from sources alternative to MRI and CT (computerized tomography) scans, a tFUS adapted to deliver ultrasonic energy based on the imaging data provides cost-effective alternatives to systems that require expensive MRI scanners or neuro-navigation systems to accurately guide a tFUS. These technical advantages provide one or more technological advancements over prior art approaches.

1. In various embodiments, a trans-cranial focused ultrasound system (tFUS) apparatus comprises a set of at least two transducer elements that produce ultrasonic stimulation, and ultrasonic guidance means or optical guidance means that guide the ultrasonic stimulation.

2. The tFUS apparatus of clause 1, where the ultrasonic guidance means includes skull aberration correction.

3. The tFUS apparatus of clause 1 or 2, further comprising an ultrasonic transducer that provides the ultrasonic guidance means, wherein the ultrasonic stimulation comprises an ultrasonic stimulation beam that employs the ultrasonic transducer.

4. The tFUS apparatus of any of clauses 1-, wherein a first ultrasonic transducer in the set of at least two transducer elements operating within a first frequency range provides an ultrasonic stimulation beam, and a second ultrasonic transducer operating within a second frequency range provides the ultrasonic guidance means.

5. The tFUS apparatus of any of clauses 1-4, where a first ultrasonic transducer in the set of at least two transducer elements provides ultrasonic stimulation via an ultrasonic stimulation beam, and a second ultrasonic transducer provides the ultrasonic guidance means, and the first and second ultrasonic transducers are positioned on temporal windows of a head of a subject.

6. The tFUS apparatus of any of clauses 1-5, where a first transducer, positioned at a back of a head of a subject, produces the ultrasonic stimulation within a conical volume, and a second transducer, positioned on the head of a subject within a temporal window, receives a signal stimulated by the first transducer.

7. The tFUS apparatus of any of clauses 1-6, where the ultrasonic guidance means or the optical guidance means guides the ultrasonic stimulation via ultrasonic imaging in a non-linear imaging mode.

8. The tFUS apparatus of any of clauses 1-7, where the non-linear imaging mode comprises a second harmonic for guidance, and the non-linear imaging mode suppresses clutter at a fundamental frequency.

9. The tFUS apparatus of any of clauses 1-8, where ultrasonic guidance means or optical guidance means uses strain imaging when guiding the ultrasonic stimulation.

10. The tFUS apparatus of any of clauses 1-9, further comprising a processor coupled to the set of transducer elements, the processor comparing ultrasonic data received in response to the ultrasonic stimulation with a priori data to identify targets.

11. The tFUS apparatus of any of clauses 1-10, where the a priori data includes a head atlas produced by at least one of a magnetic resonance imaging (MRI) scan or a computerized tomography (CT) scan.

12. The tFUS apparatus of any of clauses 1-11, where the processor determines that a correlation between the received ultrasonic data and the head atlas has not been achieved, and blocks a procedure on a subject associated with the received ultrasonic data.

13. The tFUS apparatus of any of clauses 1-12, where ultrasonic guidance means or optical guidance means comprises an ultrasonic transducer using ultrasonic imaging to guide the ultrasonic stimulation, and the guidance provided to the ultrasonic stimulation is adapted to a motion between the ultrasonic transducer and a head of a subject.

14. The tFUS apparatus of any of clauses 1-13, where the ultrasonic transducer includes an optical registration feature detectable by the ultrasonic guidance means or the optical guidance means.

15. The tFUS apparatus of any of clauses 1-4, further comprising a light detection and ranging (LIDAR) system that provides optical imaging.

16. The tFUS apparatus of any of clauses 1-15, where a mobile phone includes the LiDAR system.

17. The tFUS apparatus of any of clauses 1-16, where the mobile phone is configured to create a mesh representation of a head of a subject using the LiDAR system.

18. The tFUS apparatus of any of clauses 1-17, further comprising a processor that, based on the mesh representation, adapts data derived from a predetermined brain structure.

19. The tFUS apparatus of any of clauses 1-18, where the processor, based the adapted data, positions the set of transducer elements.

20. The tFUS apparatus of any of clauses 1-19, where the set of at least two transducer elements includes a pair of contralateral transducers.

21. The tFUS apparatus of any of clauses 1-20, further comprising a processor that uses the pair of contralateral transducers to image a head shape of a subject.

22. The tFUS apparatus of any of clauses 1-21, further comprising one or more sensors that acquire echo data associated with a head of a subject, where a skull thickness is measured via the echo data.

23. The tFUS apparatus of any of clauses 1-22, further comprising a processor, where the ultrasonic guidance means or the optical guidance means acquire ultrasonic imaging or optical imaging, the processor uses the ultrasonic imaging or the optical imaging to obtain control points in a search space, and the processor executes a placement algorithm to operate on the control points.

Any and all combinations of any of the claim elements recited in any of the claims and/or any elements described in this application, in any fashion, fall within the contemplated scope of the present invention and protection.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

Aspects of the present embodiments may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “module,” a “system,” or a “computer.” In addition, any hardware and/or software technique, process, function, component, engine, module, or system described in the present disclosure may be implemented as a circuit or set of circuits. Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Aspects of the present disclosure are described above with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine. The instructions, when executed via the processor of the computer or other programmable data processing apparatus, enable the implementation of the functions/acts specified in the flowchart and/or block diagram block or blocks. Such processors may be, without limitation, general purpose processors, special-purpose processors, application-specific processors, or field-programmable gate arrays.

The flowchart and block diagrams in the figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While the preceding is directed to embodiments of the present disclosure, other and further embodiments of the disclosure may be devised without departing from the basic scope thereof, and the scope thereof is determined by the claims that follow.

What is claimed is:

1. A trans-cranial focused ultrasound system (tFUS) apparatus, comprising:
a set of at least two transducer elements that produce ultrasonic stimulation; and
ultrasonic guidance means or optical guidance means that guide the ultrasonic stimulation.

2. The tFUS apparatus of claim 1, wherein the ultrasonic guidance means includes skull aberration correction.

3. The tFUS apparatus of claim 1, further comprising:
an ultrasonic transducer that provides the ultrasonic guidance means,
wherein the ultrasonic stimulation comprises an ultrasonic stimulation beam that employs the ultrasonic transducer.

4. The tFUS apparatus of claim 1, wherein:
a first ultrasonic transducer in the set of at least two transducer elements operating within a first frequency range provides an ultrasonic stimulation beam; and
a second ultrasonic transducer operating within a second frequency range provides the ultrasonic guidance means.

5. The tFUS apparatus of claim 1, wherein:
a first ultrasonic transducer in the set of at least two transducer elements provides ultrasonic stimulation via an ultrasonic stimulation beam; and
a second ultrasonic transducer provides the ultrasonic guidance means; and
the first and second ultrasonic transducers are positioned on temporal windows of a head of a subject.

6. The tFUS apparatus of claim 1 wherein:
a first transducer, positioned at a back of a head of a subject, produces the ultrasonic stimulation within a conical volume; and
a second transducer, positioned on the head of a subject within a temporal window, receives a signal stimulated by the first transducer.

7. The tFUS apparatus of claim 1, wherein the ultrasonic guidance means or the optical guidance means guides the ultrasonic stimulation via ultrasonic imaging in a non-linear imaging mode.

8. The tFUS apparatus of claim 7, wherein:
the non-linear imaging mode comprises a second harmonic for guidance; and
the non-linear imaging mode suppresses clutter at a fundamental frequency.

9. The tFUS apparatus of claim 1 wherein ultrasonic guidance means or optical guidance means uses strain imaging when guiding the ultrasonic stimulation.

10. The tFUS apparatus of claim 1, further comprising a processor coupled to the set of transducer elements, the processor comparing ultrasonic data received in response to the ultrasonic stimulation with a priori data to identify targets.

11. The tFUS apparatus of claim 10, wherein the a priori data includes a head atlas produced by at least one of a magnetic resonance imaging (MRI) scan or a computerized tomography (CT) scan. 12 The tFUS apparatus of claim 11, wherein the processor:
determines that a correlation between the received ultrasonic data and the head atlas has not been achieved; and
blocks a procedure on a subject associated with the received ultrasonic data.

13. The tFUS apparatus of claim 1, wherein:
ultrasonic guidance means or optical guidance means comprises an ultrasonic transducer using ultrasonic imaging to guide the ultrasonic stimulation; and
the guidance provided to the ultrasonic stimulation is adapted to a motion between the ultrasonic transducer and a head of a subject.

14. The tFUS apparatus of claim 13, wherein the ultrasonic transducer includes an optical registration feature detectable by the ultrasonic guidance means or the optical guidance means.

15. The tFUS apparatus of claim 1, further comprising a light detection and ranging (LiDAR) system that provides optical imaging.

16. The tFUS apparatus of claim 15, wherein a mobile phone includes the LiDAR system.

17. The tFUS apparatus of claim 16, wherein the mobile phone is configured to create a mesh representation of a head of a subject using the LiDAR system.

18. The tFUS apparatus of claim 17, further comprising a processor that, based on the mesh representation, adapts data derived from a predetermined brain structure.

19. The tFUS apparatus of claim 18, wherein the processor, based the adapted data, positions the set of transducer elements.

20. The tFUS apparatus of claim 1, wherein the set of at least two transducer elements includes a pair of contralateral transducers.

21. The tFUS apparatus of claim 20, further comprising a processor that uses the pair of contralateral transducers to image a head shape of a subject.

22. The tFUS apparatus of claim 1, further comprising one or more sensors that acquire echo data associated with a head of a subject, wherein a skull thickness is measured via the echo data.

23. The tFUS apparatus of claim 1, further comprising a processor, wherein:
the ultrasonic guidance means or the optical guidance means acquire ultrasonic imaging or optical imaging;
the processor uses the ultrasonic imaging or the optical imaging to obtain control points in a search space; and
the processor executes a placement algorithm to operate on the control points.
